(19) United States
(12) Patent Application Publication
(10) Pub. No.: US 2024/0130892 A1
(43) Pub. Date: Apr. 25, 2024

(54) NOVEL AQUEOUS HUMOR DRAINAGE DEVICE FOR CONTROLLING INTRAOCULAR PRESSURE

(71) Applicant: TMD LAB CO. LTD, Seoul (KR)

(72) Inventors: Chan Yun KIM, Seoul (KR); Hyoung Won BAE, Seoul (KR); Wungrak CHOI, Seoul (KR)

(21) Appl. No.: 18/269,070

(22) PCT Filed: Jun. 8, 2021
(86) PCT No.: PCT/KR2021/007133

§ 371 Date: Jun. 22, 2023

(30) Foreign Application Priority Data
Dec. 23, 2020 (KR) 10-2020-0181601
Jun. 7, 2021 (KR) 10-2021-0073792

(51) Int. Cl. A61F 9/007

(52) U.S. Cl. CPC A61F 9/00781; A61F 2210/0014; A61F 2240/001

(57) ABSTRACT

Glaucoma is a type of eye disease caused by high intraocular pressure (IOP). An increase in IOP causes damage to the appearance and function of the optic nerve and, when left untreated, leads to vision loss. However, once inserted, the conventional aqueous humor drainage devices implanted to control IOP continuously discharge a certain amount of aqueous humor regardless of how high or low the IOP is and thus have difficulty in effectively controlling the IOP. The present disclosure, which has been devised to address the above problems, relates to a novel aqueous humor drainage device for controlling IOP. The aqueous humor drainage device of the present disclosure, which is a double-layer aqueous humor drainage device having a tube made of a shape memory polymer included therein, can control both low IOP and high IOP and thus has an excellent effect of maintaining IOP within a clinically acceptable range.

### TECHNICAL FIELD

The present disclosure relates to a novel aqueous humor drainage device for controlling intraocular pressure.

This application is based on and claims priority to Korean Patent Application No. 10-2020-0181601, filed on Dec. 23, 2020 in the Korean Intellectual Property Office, and Korean Patent Application No. 10-2021-0073792, filed on Jun. 7, 2021 in the Korean Intellectual Property Office, the disclosures of which are incorporated herein by reference.

### BACKGROUND ART

Glaucoma is a type of eye disease caused by high intraocular pressure (IOP). An increase in IOP causes damage to the appearance and function of the optic nerve and, when left untreated, leads to vision loss. The increase in IOP occurs due to aqueous humor, which is a liquid that supplies nutrients and transports waste products (metabolites) while filling the eye, not flowing normally or being produced in an abnormally large amount.

Methods of treating glaucoma include applying or taking an IOP-lowering agent as a drug or glaucoma filtration surgery in which a small hole is made in the iris with a laser to help circulation and discharge of aqueous humor. However, the drug may be inconvenient due to periodic administration and may have side effects such as hypertension, respiratory trouble, kidney stones, and death, and in the case of the laser treatment, it is known that a therapeutic effect of the laser treatment is not sufficient for the laser treatment to completely replace glaucoma surgery, and the effect of laser trabeculoplasty decreases over time. In a case in which the drug or laser treatment has failed or in order to prevent an increase in IOP after the drug treatment and filtration, surgery is performed to insert an aqueous humor drainage device, configured to control the amount of aqueous humor to maintain IOP at a certain level, into the eye. However, most of the conventional aqueous humor drainage devices are tubular devices having a diameter of a certain size and, once inserted, continuously discharge a certain amount of aqueous humor regardless of how high or low the IOP is, making it difficult to effectively control the IOP. Also, in general, when glaucoma surgery is performed, ocular hypotension, in which IOP rapidly decreases, occurs immediately after the surgery, and ocular hypertension, in which IOP rises again, occurs a certain amount of time after the surgery. The conventional aqueous humor drainage devices only serve to decrease or increase IOP, and there is no aqueous humor drainage device that can perform control for both low IOP and high IOP.

Accordingly, the present disclosure, which has been devised to address the above problems, relates to a novel aqueous humor drainage device for controlling IOP. The aqueous humor drainage device of the present disclosure, which is a double-layer aqueous humor drainage device having a tube made of a shape memory polymer included therein, can, by controlling the expansion of an inner diameter to vary according to IOP, control both low IOP and high IOP without rapidly decreasing or increasing IOP and thus has an effect of maintaining IOP within a clinically acceptable range. Therefore, the aqueous humor drainage device of the present disclosure is expected to be widely used in the medical and health fields.

### DISCLOSURE

### Technical Problem

The present disclosure is directed to providing a novel aqueous humor drainage device for controlling intraocular pressure (IOP).

However, objectives to be achieved by the present disclosure are not limited to that mentioned above, and other unmentioned objectives should be clearly understood by those of ordinary skill in the art from the description below.

### Technical Solution

Hereinafter, various embodiments of the present disclosure will be described with reference to the accompanying drawings. In the following description, various specific details such as specific shapes, compositions, and processes are described for complete understanding of the present disclosure. However, a specific embodiment may be carried out without one or more of these specific details or may be carried out together with other known methods and forms. In another example, known processes and manufacturing techniques are not described as specific details in order to not unnecessarily obscure the present disclosure. Throughout this specification, reference to “one embodiment” or “an embodiment” means that a particular feature, shape, composition, or characteristic described in connection with the embodiment can be included in one or more embodiments of the present disclosure. Accordingly, the appearance of the phrase “in one embodiment” or “in an embodiment” in various places in this specification does not necessarily refer to the same embodiment of the present disclosure. In addition, a particular feature, shape, composition, or characteristic may be combined in any suitable way in one or more embodiments.

Unless particularly defined otherwise in this specification, all scientific and technical terms used herein have the same meanings as those generally understood by those of ordinary skill in the art to which the present disclosure pertains.

Glaucoma is a type of eye disease caused by high intraocular pressure (IOP). An increase in IOP causes damage to the appearance and function of the optic nerve and, when left untreated, leads to vision loss. The increase in IOP occurs due to aqueous humor (the fluid which is produced by the eye, maintains the shape of the eye, and supplies the eye with nutrients), which is a liquid that supplies nutrients and transports waste products (metabolites) while filling the eye, not flowing normally or being produced in an abnormally large amount.

Aqueous humor is produced in an amount of about 2 to 3 μl per minute in the ciliary body and exits the eye through the iridocorneal angle through a system consisting of the trabecular meshwork, the Schlemm's canal, collector channel, an episcleral vein, and a conjunctival vein. In particular, aqueous humor is discharged to the outside of the eye through about two outflow paths. The first outflow path is a uveoscleral outflow path through which aqueous humor is absorbed into a blood vessel via the ciliary body and the sclera, and the second outflow path is a trabecular outflow path through which aqueous humor is absorbed into a venous layer via the trabecular meshwork and the Schlemm's canal. The second outflow path through the trabecular meshwork is the main path for the drainage of aqueous humor. Generally, the speed of formation of aqueous humor is equal to the speed of outflow of aqueous humor, and thus IOP is kept almost constant within a range of 15 to 21 mmHg. However, in the case of glaucoma, resistance to the outflow through the trabecular meshwork is abnormally high.

In the case of primary open-angle glaucoma which is the most common form of glaucoma and accounts for 60 to 90% of all glaucoma cases, resistance is present along an outer surface of the trabecular meshwork and an inner wall of the Schlemm's canal, and it is known that the aqueous humor outflow rate decreases due to reasons such as trabecular occlusion caused by foreign matter, loss of trabecular endothelial cells, reduction in trabecular outflow rate, and loss of phagocytic power which is a normal trabecular meshwork function. As a result, IOP increases, and the increased IOP compresses the axon of the optic nerve or causes an abnormal blood flow in the optic nerve, which may lead to increased visual field damage or blindness.

IOP is the most clearly identified risk factor among several risk factors associated with the development of glaucoma, and control of IOP is still the most reliable way to treat glaucoma. To date, IOP is reduced to treat glaucoma, and generally, drug treatment or laser treatment is given priority, and when the drug treatment or laser treatment fails to reduce IOP, surgery is performed. However, drugs, which are classified into eye drops and oral medications and serve to suppress the production of aqueous humor or increase the outflow of aqueous humor, may be inconvenient due to periodic administration and may have side effects such as hypertension, respiratory trouble, kidney stones, and death, and in the case of laser treatment, it is known that a therapeutic effect of the laser treatment is not sufficient for the laser treatment to completely replace glaucoma surgery, and the effect of laser trabeculoplasty decreases over time. According to one study, IOP control for ten years failed in 95% of patients who received argon laser trabeculoplasty. In the case of surgery, a trabeculectomy is performed, in which the back of the conjunctiva is dissected to expose the scleral border and create a scleral flap, and the scleral flap is sutured loosely to allow aqueous humor to flow downward through an opening of the scleral flap and collect in a high space under the conjunctiva. That is, this is a principle that IOP is controlled through transconjunctival filtration in which aqueous humor comes out from the anterior chamber and, from around an edge of a scleral strip, enters a subconjunctival space and passes through the conjunctiva, absorption of aqueous humor into the lymphatic system, and absorption of aqueous humor by blood vessels of subconjunctival tissue. Glaucoma filtration surgery has a success rate of 70 to 90% in the case of primary open-angle glaucoma, pigmentary glaucoma, and pseudoexfoliation glaucoma but has a remarkably low success rate in the case of neovascular glaucoma, secondary glaucoma due to uveitis, aphakic or pseudophakic glaucoma, and revision surgery performed in patients in which the conventional filtration surgery has previously failed.

The present disclosure provides a novel aqueous humor drainage device for IOP control in an eye disease accompanied by failure of IOP control.

The eye disease may include glaucoma caused by an increase in IOP, and the glaucoma may include congenital glaucoma, traumatic glaucoma, suspected glaucoma, ocular hypertension, primary open-angle glaucoma, normal tension glaucoma, capsular glaucoma with pseudoexfoliation of lens, chronic simple glaucoma, low tension glaucoma, pigmentary glaucoma, primary closed-angle glaucoma, acute closed-angle glaucoma, chronic closed-angle glaucoma, intermittent closed-angle glaucoma, glaucoma secondary to eye trauma, glaucoma secondary to eye inflammation, glaucoma secondary to drugs, neovascular glaucoma, secondary glaucoma due to uveitis, or the like.

The aqueous humor drainage device of the present disclosure may be manufactured to have a double-tube structure in which a first tube (shape memory polymer (SMP) tube) is inserted into a second tube (silicone tube).

The SMP, which is a PCL-co-PGMA copolymer, may be prepared by mixing 78 to 90 mol % of ε-caprolactone (CL), 10 to 22 mol % of glycidyl methacrylate (GMA), 1 to 2.2 mol % of 1,6-hexanediol (HD), 1 mol % of 1,5,7-triazabicyclo[4.4.0]dec-5-ene (TBD), and 0.5 mol % of hydroquinone (HQ) and is represented by Chemical Formula 1 below.

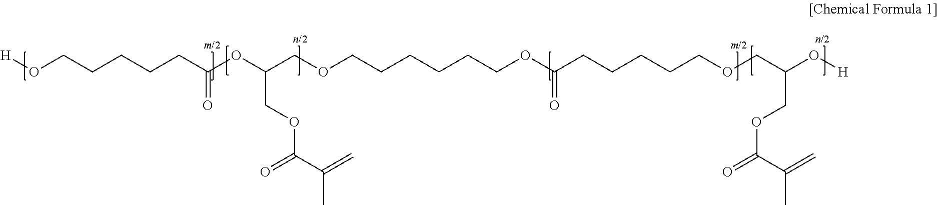

In Chemical Formula 1 above, m and n represent mol % of repeat units, m+n may be 100, and m may be in a range of 80 to 95 or 88 to 94. Here, mol % refers to fractions of the repeat units of m and n and, specifically, may refer to molar fractions thereof. For example, in PCL-co-PGMA, mol % may refer to molar fractions of repeat units of poly(ε-caprolactone) (PCL) and poly(glycidyl methacrylate) (PGMA).

Also, the SMP may be crosslinked by irradiation and may be controlled to have a melting temperature (Tm) in a range of 35° C. to 40° C.

The first tube may have a length of 3 mm and an inner diameter of 0.05 mm, and the second tube may have a length of 10 mm and an inner diameter of 0.305 mm. The lengths and inner diameters of the first tube and the second tube may be controlled in a range of ±10%, and in a case in which the material, length, and inner diameter of each of the first tube and the second tube are designed in conditions stated above, the aqueous humor drainage device of the present disclosure may control an IOP of 0 mmHg to 30 mmHg to be in a range of 5 mmHg to 10 mmHg.

One embodiment of the present disclosure provides an aqueous humor drainage device including a first tube and a second tube, wherein the first tube is positioned inside the second tube, the first tube is made of a shape memory polymer (SMP), the SMP is a copolymer of ε-caprolactone and glycidyl methacrylate, the SMP is represented by Chemical Formula 1 below, the first tube is manufactured to have a length in a range of 2.7 to 3.3 mm and an inner diameter in a range of 0.045 to 0.055 mm, the second tube is manufactured to have a length in a range of 9 to 11 mm and an inner diameter in a range of 0.2745 to 0.3355 mm, the aqueous humor drainage device is for intraocular pressure (IOP) control in an eye disease accompanied by failure of IOP control, the eye disease may be glaucoma, the glaucoma may be any one or more selected from the group consisting of congenital glaucoma, traumatic glaucoma, suspected glaucoma, ocular hypertension, primary open-angle glaucoma, normal tension glaucoma, capsular glaucoma with pseudoexfoliation of lens, chronic simple glaucoma, low tension glaucoma, pigmentary glaucoma, primary closed-angle glaucoma, acute closed-angle glaucoma, chronic closed-angle glaucoma, intermittent closed-angle glaucoma, glaucoma secondary to eye trauma, glaucoma secondary to eye inflammation, glaucoma secondary to drugs, neovascular glaucoma, and secondary glaucoma due to uveitis, the aqueous humor drainage device is able to control both low IOP and high IOP, and the aqueous humor drainage device controls an IOP of 0 mmHg to 30 mmHg to be in a range of 5 mmHg to 10 mmHg.

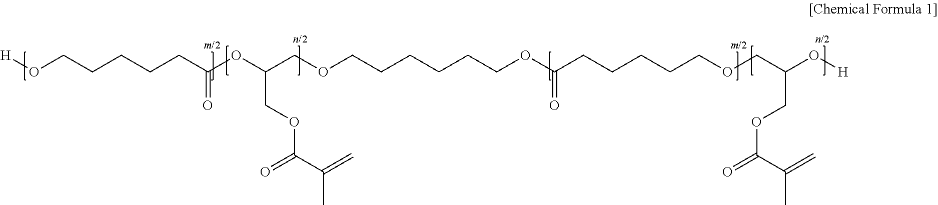

In Chemical Formula 1 above, m and n represent mol % of repeat units, m+n may be 100, and m may be in a range of 80 to 95 or 88 to 94. Here, mol % refers to fractions of the repeat units of m and n and, specifically, may refer to molar fractions thereof. For example, in PCL-co-PGMA, mol % may refer to molar fractions of repeat units of poly(ε-caprolactone) (PCL) and poly(glycidyl methacrylate) (PGMA). Each step of the present disclosure will be described in detail below.

### Advantageous Effects

A novel aqueous humor drainage device for intraocular pressure (IOP) control according to the present disclosure, which is a double-layer aqueous humor drainage device having a tube made of a shape memory polymer (SMP) included therein, can, by controlling the expansion of an inner diameter to vary according to IOP, control both low IOP and high IOP without rapidly decreasing or increasing IOP and thus has an effect of maintaining IOP within a clinically acceptable range. Therefore, the aqueous humor drainage device of the present disclosure is expected to be widely used in the medical and health fields.

### DESCRIPTION OF DRAWINGS

FIG. 1 is a schematic diagram illustrating an aqueous humor drainage device (with a shape memory polymer (SMP) tube) according to one embodiment of the present disclosure.

FIG. 2 shows a result of designing a length, a flow velocity, and an inner diameter of the aqueous humor drainage device (with the SMP tube) through computational fluid dynamics modeling according to one embodiment of the present disclosure.

FIG. 3 shows a result of measuring a melting temperature (Tm) and a dynamic contact angle (DCA) of a copolymer of poly(ε-caprolactone) (PCL) and poly(glycidyl methacrylate) (PGMA) according to one embodiment of the present disclosure.

FIG. 4 shows a result of designing an inner diameter and an outer diameter of a first tube (SMP tube) in consideration of deformation thereof according to temperature and measuring a change in the inner diameter and the outer diameter when the tube elongates according to one embodiment of the present disclosure.

FIG. 5 shows a result of measuring a pressure control effect of the aqueous humor drainage device (with the SMP tube) as compared to an aqueous humor drainage device (without the SMP tube) according to one embodiment of the present disclosure.

FIG. 6 shows a result of measuring pressure when the elongated first tube is restored to its original shape according to one embodiment of the present disclosure.

FIG. 7 shows a result of measuring in-vivo cell compatibility and a pressure control effect of the aqueous humor drainage device according to one embodiment of the present disclosure.

### MODES OF THE INVENTION

Hereinafter, the present disclosure will be described in more detail using examples. These examples are only for describing the present disclosure in more detail, and it should be apparent to those of ordinary skill in the art that the scope of the present disclosure is not limited by these examples according to the gist of the present disclosure.

Throughout the specification, when a certain part is described as “including” a certain component, unless specifically stated otherwise, the certain part may further include another component instead of excluding other components.

### Example 1. Manufacture of Aqueous Humor Drainage Device

### Example 1-1. Preparation of Shape Memory Polymer (SMP)

A ε-caprolactone (CL) monomer and a glycidyl methacrylate (GMA) monomer were copolymerized, and as a catalyst, 1,5,7-triazabicyclo[4.4.0]dec-5-ene (TBD), tin(II)(2-ethylhexanoate), trimethylopropane tris(3-mercaptopropionate), or zinc succinate, preferably, 1,5,7-triazabicyclo[4.4.0]dec-5-ene (TBD), was used. In this way, a PCL-co-PGMA SMP represented by Chemical Formula 1 below was prepared. 1,5,7-triazabicyclo[4.4.0]dec-5-ene (TBD) is a material for inducing simultaneous ring-opening polymerization of the two monomers CL and GMA and has an effect of reducing the time taken for synthesis of SMP. A use amount of the catalyst is not limited, but the catalyst may be used at a concentration of 0.5 to 1 mol relative to starting materials.

At the time when the polymerization conversion rate is almost zero, that is, during an initial reaction, a 1,6-hexanediol (HD) initiator and a polymerization inhibitor may be simultaneously added before the GMA monomer is added. In this way, a reaction between GMA acrylic groups sensitive to temperature can be inhibited. The polymerization inhibitor serves to inhibit an exothermic reaction locally occurring at a later stage of polymerization and remove unreacted remaining radicals to end the reaction. The polymerization inhibitor is not particularly limited, but one or more selected from the group consisting of hydroquinone (HQ), hydroquinone monomethyl ether, p-benzoquinone, and phenothiazine may be used as the polymerization inhibitor.

The preparation of the SMP may be performed at an average temperature of 80 to 140° C. or 100 to 130° C., but in a case in which polymer synthesis is performed at a temperature lower than 100° C., catalysis may not proceed, and when polymer synthesis is performed at a temperature higher than 130° C., the catalytic reaction rate may decrease.

The preparation of the SMP may further include inducing a photo-crosslinking reaction of the SMP. By inducing the photo-crosslinking reaction of the SMP, the melting point of the SMP may be lowered. In the present disclosure, the SMP was crosslinked by irradiation.

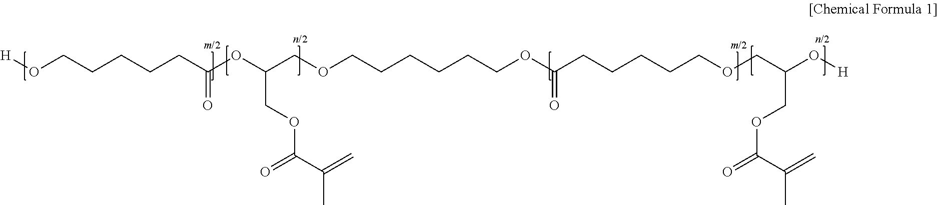

In Chemical Formula 1 above, m and n represent mol % of repeat units, m+n may be 100, and m may be in a range of 80 to 95 or 88 to 94. Here, mol % refers to fractions of the repeat units of m and n and, specifically, may refer to molar fractions thereof. For example, in PCL-co-PGMA, mol % may refer to molar fractions of repeat units of poly(ε-caprolactone) (PCL) and poly(glycidyl methacrylate) (PGMA).

Specifically, in a preparation method of Chemical Formula 1 above, in order to prepare the PCL-co-PGMA SMP in which 88 to 94 mol % of PCL and 6 to 12 mol % of PGMA are polymerized, 78 to 90 mol % of CL, 10 to 22 mol % of GMA, 1 to 2.2 mol % of HD, 1 mol % of TBD, and 0.5 mol % of HQ were mixed in a glass reactor, and when temperature inside the glass reactor in which the two monomers were mixed was determined as being thermally stable, as a catalyst for inducing simultaneous ring-opening polymerization of CL and GMA, TBD (1 mmol, 140 mg) was dissolved in 1 ml of acetonitrile and then added into the glass reactor, and the solution in the glass reactor was stirred at 110° C. for 2 hours. All processes were carried out under high-purity nitrogen. After the reaction, the product was dissolved in 10 ml of chloroform, and the resulting product was slowly dropped into diethyl ether (400 ml) and precipitated therein. Next, the precipitate was filtered through filter paper, a solvent was removed through a rotary evaporator, and the resulting product was dried under reduced pressure to synthesize the PCL-co-PGMA SMP.

### Example 1-2. Manufacture of Aqueous Humor Drainage Device

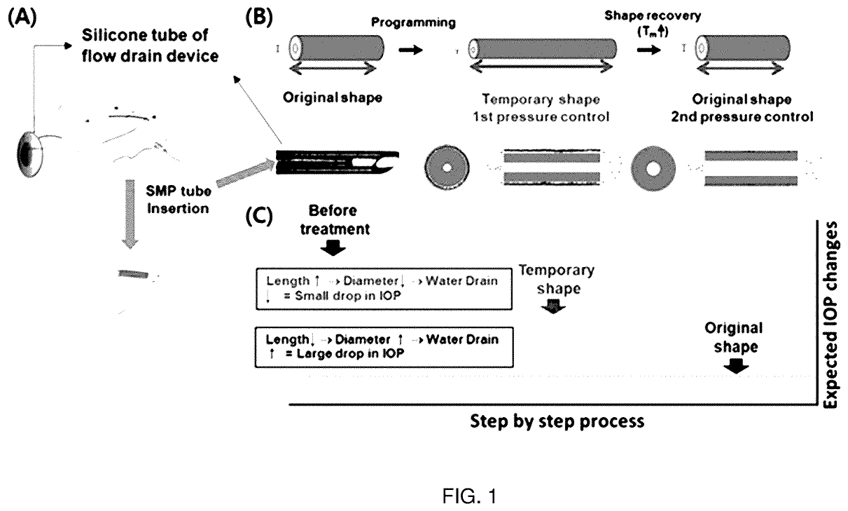

A first tube (SMP tube) was made using the SMP of Example 1-1 above, and the first tube (SMP tube) was inserted into a second tube (silicone tube) to manufacture an aqueous humor drainage device having a double-tube structure. A schematic diagram of the aqueous humor drainage device of the present disclosure is shown in FIG. 1. Hereinafter, the aqueous humor drainage device of the present disclosure in which the first tube is inserted into the second tube will be referred to as “w/SMP tube,” and an aqueous humor drainage device of the control group in which the first tube is not inserted into the second tube will be referred to as “w/o SMP tube.” In the w/SMP tube, an outer diameter of the first tube may be reduced in order to facilitate the process of inserting the first tube into the second tube, and an inner diameter of the first tube may be reduced to finely control intraocular pressure (IOP) at an early stage of treatment (FIG. 1A). In IOP control, programming the w/SMP tube to temporarily have an elongated shape reduces the inner diameter and reduces the amount of drained eye fluid, thus causing a slight drop in IOP (FIGS. 1B and 1C), and when the w/SMP tube is treated with warm water having a temperature of about 37° C., the w/SMP tube reacts to a temperature change until a melting temperature (Tm), and the shape of the w/SMP tube is restored from the temporary shape to a circular shape (the original shape), causing a further significant drop in IOP. Through such a process, IOP reduction may be customized and applied according to the treatment stage, disease progression rate, and patient condition. In general, body fluids have a temperature of about 34.72° C., which is lower than body temperature (37° C.). Therefore, the w/SMP tube of the present disclosure was designed so that temperature thereof can be controlled within a range of 32 to 44° C.

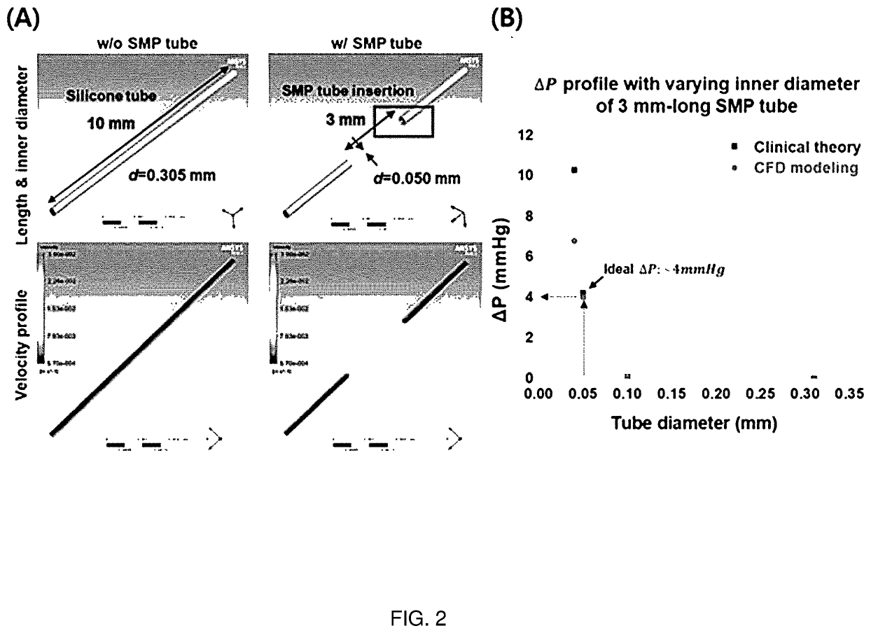

The length and flow velocity of the w/SMP tube and the inner diameter of the circular shape of the w/SMP tube were calculated through computational fluid dynamics modeling, which is shown in FIG. 2. In order to achieve the best therapeutic effect in the initial stage of maintaining IOP in glaucoma patients, a pressure difference (ΔP) between an inlet (eyeball) and an outlet (drainage) of the implanted aqueous humor drainage device should be the same. Therefore, a fluid dynamics model assumed a situation in which the first tube with a length of 3 mm was inserted into the second tube with a length of 10 mm and an inner diameter of 0.305 mm (top of FIG. 2A). As a result, while an inner diameter of 0.305 mm and IOP of 0.01 mmHg or lower were maintained in the w/o SMP tube in which the first tube was not inserted, in the w/SMP tube in which the first tube was inserted, the inner diameter reduced from 0.305 mm to 0.100 mm and further to 0.050 mm, and the IOP increased from 0.01 mmHg or less to 0.25 mmHg (when the inner diameter was 0.100 mm) and further to 4 mmHg (when the inner diameter was 0.05 mm). Accordingly, for the IOP to reach the parameter 4 mmHg (ΔP), the first tube was manufactured to have a length of 3 mm and an inner diameter of 0.05 mm (FIG. 2B).

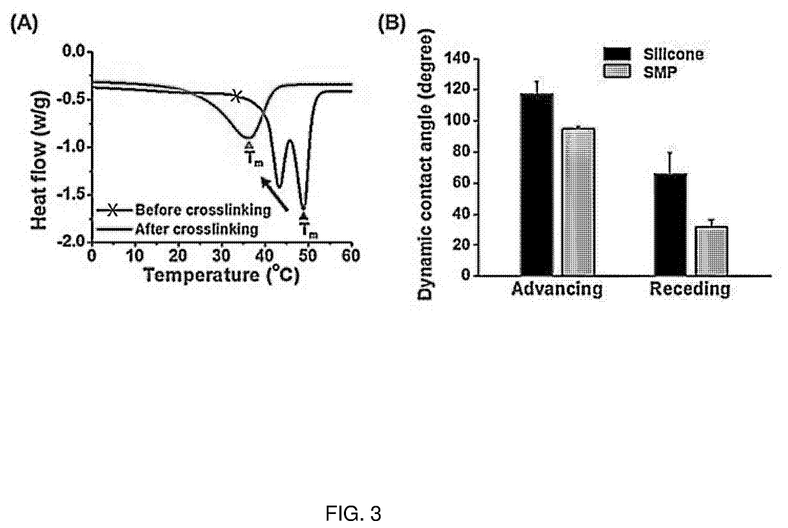

Two essential functions for stably using the first tube are allowing shape recovery at body temperature and increasing drainage efficiency by activating surface-mediated adsorption of moisture. In that sense, in the first tube crosslinked based on PCL, the Tm (shape recovery temperature) was reduced at about 50° C. close to body temperature. Although crosslinking is generally known to form a polymer network and increase the Tm, crosslinking sometimes interferes with crystallinity due to a change in chain structure. The Tm of the PCL-based first tube was reduced due to excellent inhibition of crystallinity during crosslinking. Due to the crystalline nature of the PCL-PGMA copolymer, the melting point (Tm) of each of PCL and PGMA formed two peaks (FIG. 3A). A dynamic contact angle (DCA) was found to be lower in the first tube (SMP) than in the second tube (silicone) in advancing and receding (FIG. 3B).

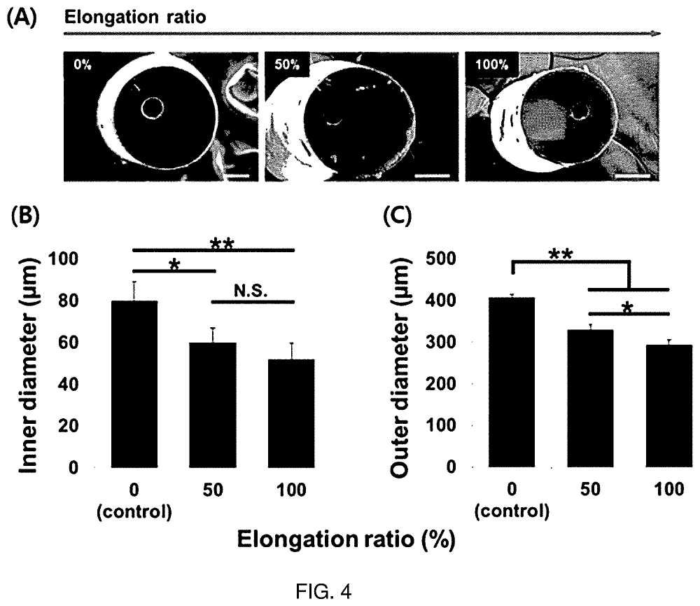

Also, in consideration of deformation according to temperature, the first tube was manufactured to have a maximum outer diameter of 0.305 mm to allow close contact with the second tube and a maximum inner diameter of 0.05 mm to maintain the IOP up to 4 mmHg (ΔP). Results of measuring the inner diameter and outer diameter of the first tube, when the length of the first tube was elongated 50% or 100%, by using an electron microscope, are shown in FIG. 4A. As a result of analysis, the inner diameter and outer diameter greatly decreased when the length of the first tube was elongated 50%, and the outer diameter significantly decreased to 300 μm when the length of the first tube was elongated 100%. There was no significant change in the inner diameter of the first tube when the length of the first tube was elongated from 50% to 100% (FIGS. 4B and 4C). Therefore, an elongation ratio of 100% was applied in a follow-up experiment.

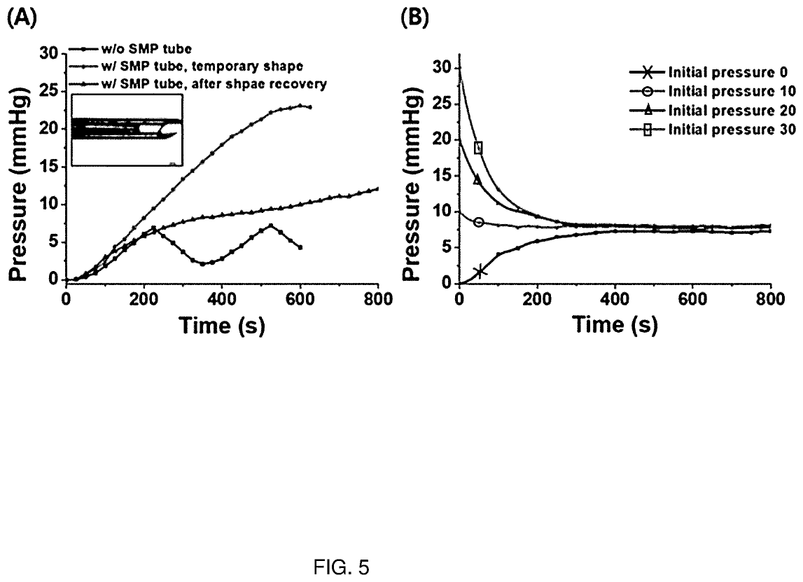

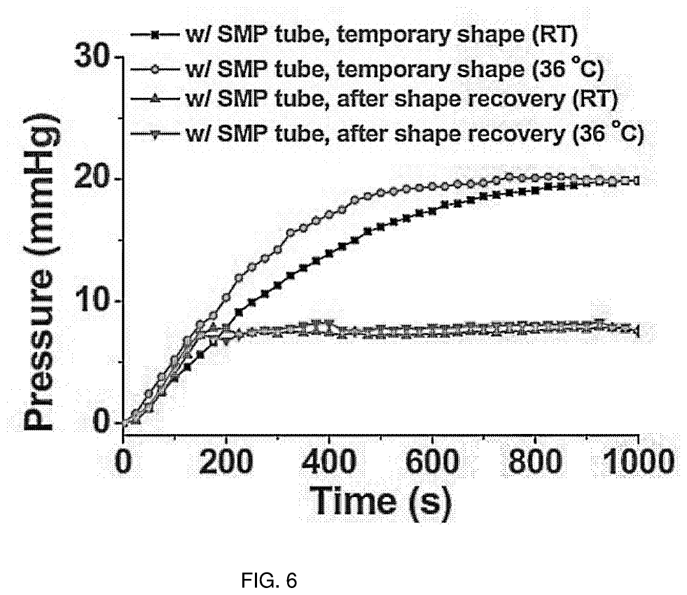

Since an outer circumferential portion of the first tube and an inner circumferential portion of the second tube come into close contact and limit circumferential expansion, diameter expansion of the first tube itself due to shape recovery should be able to be controlled. Therefore, pressure according to temperature was custom designed for a case in which the first tube is temporarily elongated (temporary shape) or restored to a circular shape (original shape) in the w/SMP tube and for a case of the w/o SMP tube. For the experiment, a 25G needle was connected to one end of the tube, and the other end of the tube was connected to a pump. This is a principle that, through pumping, distilled water flows like tears through the tube and is discharged. A pressure sensor was placed in a flow rate tube line, and a change in pressure was recorded for 800 seconds using a pressure gauge, the result of which is shown in FIG. 5. As a result of the experiment, the w/SMP tube maintained a higher pressure level than the w/o SMP tube (FIG. 5A). In the w/o SMP tube, pressure changed in a zigzag manner, and pressure measurement was not possible after 600 seconds. In the w/SMP tube having an elongated shape (temporary shape), pressure was maintained at the highest level in a stiff incremental state, and pressure increased beyond the range detectable by the pressure gauge after 600 seconds. However, as a result of testing pressure at the time of shape recovery after elongation of the first tube at room temperature, it was confirmed that after a log phase of rapid increase, a lag phase of plateau was eventually reached. (FIG. 6).

Since both high IOP in the state of glaucoma disease and a rapid drop in IOP right after glaucoma surgery should be controlled using a single aqueous humor drainage device, pressure was measured for stabilization over time in the low (0 mmHg), normal (10 to 20 mmHg), or hypertensive (30 mmHg) range. As a result, the first tube was found to maintain a pressure of 7 mmHg or lower from about 300 seconds after shape recovery regardless of a starting pressure (FIG. 5B). This shows that the aqueous humor drainage device of the present disclosure has an excellent pressure control effect for both high IOP and low IOP.

### Example 2. Confirmation of In-Vivo Effects of Aqueous Humor Drainage Device

The biocompatibility and in-vivo IOP control effect of the aqueous humor drainage device of the present disclosure manufactured in Example 1 were tested.

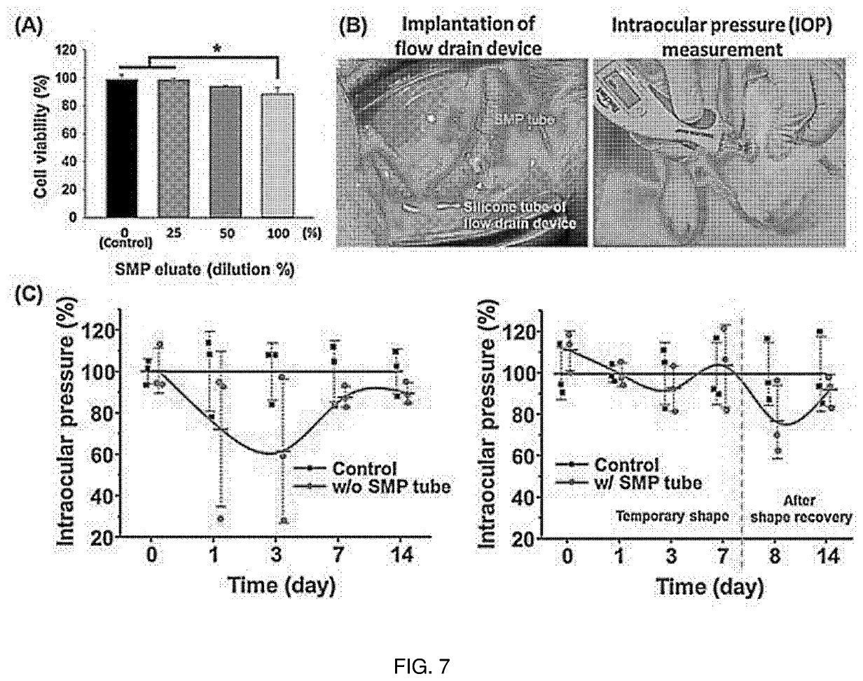

First, biocompatibility was tested by culturing rat fibroblasts (L929) for 24 hours with an eluate dilution of the aqueous humor drainage device. As a result, 80% of cells or more survived in the 100% eluate-comprising group compared to the control group (not comprising an eluate), and the aqueous humor drainage device of the present disclosure was confirmed as having no cytotoxicity (FIG. 7A).

Next, the aqueous humor drainage device (w/SMP tube) of the present disclosure was implanted into a rabbit (FIG. 7B), and IOP was measured three times each on days 1, 3, 7, and 14 after surgery (FIG. 7C). Each measured value was converted based on a normal IOP value in the same rabbit (control group) as 100%. As a result of the experiment, while the group implanted with the w/o SMP tube showed a rapid IOP drop of up to 40% continuously until day 3 and a slow insufficient recovery afterwards, the group implanted with the w/SMP tube showed complete recovery on day 7. Then, in a case in which the inner diameter of the tube was controlled through additional manipulation of the SMP, the IOP decreased again and an IOP drop of about 30% occurred until day 8, and an IOP lower than the initial IOP was maintained until day 14. In this way, the aqueous humor drainage device of the present disclosure was confirmed as being able to control both low IOP and high IOP without rapidly decreasing or increasing IOP and thus maintain the IOP within a clinically acceptable range.

Specific parts of the present disclosure have been described in detail above, but it should be apparent to those of ordinary skill in the art that the above detailed descriptions are only exemplary examples, and the scope of the present disclosure is not limited thereto. Therefore, the substantial scope of the present disclosure is defined by the appended claims and their equivalents.

### INDUSTRIAL APPLICABILITY

Glaucoma is a type of eye disease caused by high intraocular pressure (IOP). An increase in IOP causes damage to the appearance and function of the optic nerve and, when left untreated, leads to vision loss. However, once inserted, the conventional aqueous humor drainage devices implanted to control IOP continuously discharge a certain amount of aqueous humor regardless of how high or low the IOP is, making it difficult to effectively control the IOP.

The present disclosure, which has been devised to address the above problems, relates to a novel aqueous humor drainage device for controlling IOP.

The aqueous humor drainage device of the present disclosure, which is a double-layer aqueous humor drainage device having a tube made of a shape memory polymer included therein, can control both low IOP and high IOP and thus has an excellent effect of maintaining IOP within a clinically acceptable range.

What is claimed is:

1. An aqueous humor drainage device comprising:
a first tube; and
a second tube,
wherein the first tube is positioned inside the second tube, and
the first tube is made of a shape memory polymer (SMP)

2. The aqueous humor drainage device of claim 1, wherein the SMP is a copolymer of ε-caprolactone and glycidyl methacrylate.

3. The aqueous humor drainage device of claim 2, wherein the SMP is represented by Chemical Formula 1 below: 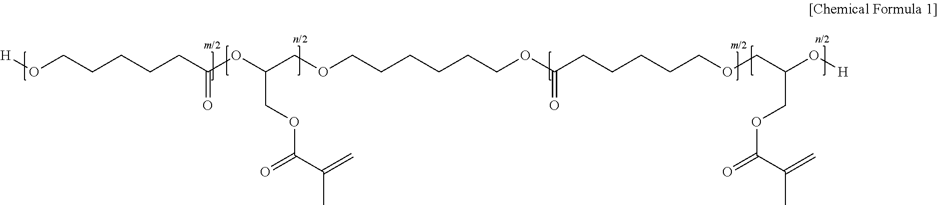
wherein, in Chemical Formula 1 above, m and n represent mol % of repeat units, m+n is 100, and m is in a range of 80 to 95.

4. The aqueous humor drainage device of claim 1, wherein the first tube is manufactured to have a length in a range of 2.7 to 3.3 mm and an inner diameter in a range of 0.045 to 0.055 mm.

5. The aqueous humor drainage device of claim 1, wherein the second tube is manufactured to have a length in a range of 9 to 11 mm and an inner diameter in a range of 0.2745 to 0.3355 mm.

6. The aqueous humor drainage device of claim 1, wherein the aqueous humor drainage device is for intraocular pressure (IOP) control in an eye disease accompanied by failure of IOP control.

7. The aqueous humor drainage device of claim 6, wherein the eye disease is glaucoma.

8. The aqueous humor drainage device of claim 7, wherein the glaucoma is any one or more selected from the group consisting of congenital glaucoma, traumatic glaucoma, suspected glaucoma, ocular hypertension, primary open-angle glaucoma, normal tension glaucoma, capsular glaucoma with pseudoexfoliation of lens, chronic simple glaucoma, low tension glaucoma, pigmentary glaucoma, primary closed-angle glaucoma, acute closed-angle glaucoma, chronic closed-angle glaucoma, intermittent closed-angle glaucoma, glaucoma secondary to eye trauma, glaucoma secondary to eye inflammation, glaucoma secondary to drugs, neovascular glaucoma, and secondary glaucoma due to uveitis.

9. The aqueous humor drainage device of claim 1, wherein the aqueous humor drainage device is able to control both low IOP and high IOP.

10. The aqueous humor drainage device of claim 9, wherein the aqueous humor drainage device controls an IOP of 0 mmHg to 30 mmHg to be in a range of 5 mmHg to 10 mmHg.
